(19) United States
(12) Patent Application Publication
(10) Pub. No.: US 2008/0279835 A1
(43) Pub. Date: Nov. 13, 2008

(54) Method of Stem Cell Therapy for Cardiovascular Repair

(75) Inventors: Robert J. Henning, Tampa, FL (US); Paul R. Sanberg, Spring Hill, FL (US)

Correspondence Address: SMITH HOPEN, PA, 180 PINE AVENUE NORTH, OLDSMAR, FL, 34677, US

(73) Assignee: UNIVERSITY OF SOUTH FLORIDA, Tampa, FL (US)

(21) Appl. No.: 12/117,197

(22) Filed: May 8, 2008

Related U.S. Application Data
(63) Continuation of application No. PCT/US2006/060692, filed on Nov. 8, 2006.
(60) Provisional application No. 60/735,027, filed on Nov. 8, 2005.

(51) Int. Cl. A61K 35/14; A61P 9/10

(52) U.S. Cl. 424/93.71

(57) ABSTRACT

A method of treating acute myocardial infarction has the steps of providing human umbilical cord blood cells (HUCBC); and administering the HUCBC to the individual with the acute myocardial infarction at particular time intervals after said myocardial infarction. Preferably the intervals are about one to about three hours or about 12 to about 48 hours after the acute myocardial infarction.

### CROSS REFERENCE TO RELATED APPLICATIONS

This application is a continuation of prior filed International Application, Serial Number PCT/US2006/060692 filed Nov. 8, 2006, which claims priority to U.S. provisional patent application No. 60/735,027 filed Nov. 8, 2005 which is hereby incorporated by reference into this disclosure.

### TECHNICAL FIELD

This invention is in the field of medical treatment, more specifically treatment of myocardial infarction with an infusion of cells derived from human umbilical cord blood.

### BACKGROUND

In industrialized countries, one of the most common diagnoses in hospitalized patients is myocardial infarction (MI), commonly known as a heart attack. In the United States alone, there are about 1.5 million MIs per year. The mortality rate is about 30%, with more than half occurring before the patient arrives at the hospital. Once the patient reaches the hospital, only about 4% die in the first year. However, among over-65 individuals, the mortality rate is 30% at one month and 35% at one year after the MI.

MI typically occurs when there is an abrupt decrease in coronary artery flow, followed by a thrombotic occlusion at a previously narrowing due to atherosclerosis. Cytokine and chemokine expression play an important role in inflammatory cell recruitment and are a prominent feature of the inflammation response with myocardial infarction. The triggers of cytokine release in MI include myocardial ischemia, reactive oxygen species, and mechanical deformation of the ventricular wall. The proinflammatory cytokine TNFα activates cytotoxic T cells and matrix metalloproteinases, induces apoptosis, depresses LV function, and ultimately causes ventricular remodeling. Serum concentrations of TNFα after acute MI correlate with collagen deposition in the heart and with increased LV end-diastolic diameter. Moreover, TNFα together with IFNγ promote induction of intercellular adhesion molecule-1 (ICAM-1), neutrophil adhesion to myocytes and endothelial cells and cell dysfunction. Conversely, in animal models of myocardial infarction in which the TNFα gene is ablated or TNFα is inhibited with antibody, there is a significant reduction in infarction size and an increase in left ventricular ejection fraction.

Monocyte chemoattractant protein-1 (MCP-1) and MIP attract monocytes to an inflammatory focus. MCP strongly attracts macrophages, T cells and natural killer cells, resulting in cytokine synthesis, reperfusion injury and formation of granulation tissue in the healing MI. MCP-1 also stimulates the expression of ICAM-1, thereby causing the adhesion of neutrophils to cardiac myocytes. In contrast, the inhibition or ablation of MCP-1 attenuates post-MI LV remodeling. MCP-1 with MIP are also critical in autoimmune myocarditis, where the inflammatory infiltrate consists of more than 70% mononuclear cells.

Interferon gamma (INFγ) with IL-1 and IL-2 stimulate TNF production by cardiac myocytes, and thereby induce myocyte apoptosis and depress cardiac function. With TNFα, INFγ increases the production of superoxide anions that react with nitric oxice (NO) to form peroxynitrite which desensitizes myofilaments to calcium and causes contractile dysfunction and heart failure. INFγ also synergizes with C-reactive protein to increase monocyte tissue factor by as much as 50-100 times, which contributes to vascular thrombosis.

Transplantation of stem cells has been proposed as a means of treating numerous diseases and conditions, including myocardial infarctions (MIs). The powerful multipotent potential of stem cells may make it possible to effectively treat diseases and injuries with complicated disruptions in neuronal physiology and function, such as MIs, in which more than one cell type is affected. Stem cells are important treatment candidates for MI and other cardiovascular diseases because of their ability to differentiate in vitro and in vivo into a variety of cells.

Recently investigators have discovered that bone marrow hematopoietic and mesenchymal stem cells have the capacity to limit myocardial infarction size and augment ventricular systolic wall thickening. However, the isolation, expansion, and preparation of bone marrow stem cells for therapeutic uses can require several days. In addition, the transplantation of allogeneic bone marrow stem cells usually requires the use of immunosuppressant drugs in the recipient which may contribute to an immature transplant stem cell phenotype and subject the host to possible complications of infection or malignancy.

Despite this great potential, an easily obtainable, abundant, safe and clinically proven source of stem cells has been elusive until recently. Umbilical cord blood contains a relatively high percentage of undifferentiated stem cells capable of differentiating into cardiomyocytes. Following intravenous delivery, human umbilical cord blood cells (HUCBC) survive and migrate into the myocardium of diseased animals and have been shown to promote functional recovery in animal models of MI.

In addition to the growing body of evidence supporting the potential of HUCBCs, there is a long and well established series of practical advantages of using HUCBC for clinical diseases. Cord blood is easily obtained with no risks to the mother or child. A blood sample is taken from the umbilical vein attached to the placenta after birth. The percentage of the undifferentiated stem cells present in the mononuclear fraction is small; but the absolute yield of stem cells may number in the thousands prior to expansion or other ex vivo manipulation, providing an easily obtainable and plentiful source. Hematopoietic stem cells from HUCB have been routinely and safely used to reconstitute bone marrow and blood cell lineages in children with malignant and nonmalignant diseases after treatment with myeloablative doses of chemoradiotherapy (Lu et al., 1996 Crit. Rev Oncol Hematol., 22(2):61-78; and Broxmeyer, Cellular Characteristics of cord blood and cord blood transplantation, In AABB Press. 1998 Bethesda, Md.). Early results indicate that a single cord blood sample provides enough hematopoietic stem cells to provide both short- and long-term engraftment. This suggests that these stem cells maintain extensive replicative capacity, which may not be true of hematopoietic stem cells obtained from other sources, such as adult bone marrow.

In addition, HUCBCs can also be easily cryopreserved following isolation. Cryopreservation of HUCBCs, accompanied by sustained good cell viability after thawing, also allows long-term storage and efficient shipment of cells from the laboratory to the clinic. Thus, this novel feature of cryopreservation gives HUCBCs a commercially distinct advantage in the design of cell-based therapeutic products. Although the duration of time that the cells may be stored with high viability upon thawing remains to be determined, it has been reported that after HUCBCs were frozen for at least 15 years, viable cells were thawed and survived transplant within animal models of injury (Broxmeyer et al., 2003 Proc Natl Acad Sci USA, 100(2):645-50).

Because HUCBC transplant recipients exhibit a low incidence and severity of graft-versus-host disease or immuno-rejection (Wagner et al., 1992 Blood, 79(7):1874-81; Gluckman et al., 1997 N Engl J. Med., 337(6):373-81), long-term immune suppression with its associated health risks may be unnecessary, making HUCBCs an ideal candidate for cell-based products. Furthermore, as the technology for banking HUCBCs improves, it is possible that autologous transplantation (i.e., transplantation of an individual's own cells back into that person's body) will be plausible. This would completely eliminate the need for immunosuppression during cellular therapy, which is utilized to prevent rejection but is a very difficult process to management successfully.

Intravenously administered HUCBCs preferentially survive and differentiate into cells in the damaged areas. While intravenous delivery of HUCBCs has promoted functional recovery in preclinical ischemia models, the behavioral improvements are only partial, leaving significant room for increments in the efficacy of these cells in vivo.

Because of the difficulty in effectively treating patients after myocardial infarction and other ischemic events, there is a need in the art for methods to enhance the treatment of ischemic and inflammatory events, particularly MI.

### BRIEF DESCRIPTION OF DRAWINGS

FIG. 1 is a photograph of the modified Boyden chemoattraction apparatus, showing the lower chamber and wells, the 25×80 mm framed polycarbonate membrane with 5 μm pore size and superimposed upper wells.

FIG. 2 is a graph of a standard curve showing relative fluorescence units compared to known cell numbers.

FIG. 3 graphs the HUCBC counts of cells that migrated toward infracted tissue from several experimental groups (time post infarction up to 96 hours). The 2-, 2.5-, and 24-hour post-infarction groups attracted significantly more cells (p<0.05) than did the control group.

FIG. 4 is a graph showing the migrated HUCBC counts divided by infarct size at time post infarction up to 96 hours. The 2, 2.5, and 24-hour post-infarctions attracted significantly (p<0.01) more HUCBC than did the one-hour group.

FIG. 5 is a graph showing the measured infarction sized in untreated hearts and hearts treated with HUCBC. The hearts treated with HUCBC within 2 hours and at 24 hours are significantly smaller than the saline-treated hearts. IA/TLVA indicates infarctin area divided by total left ventricular area. The asterisk indicates a comparison with 2-hr saline treatment. The double plus signs indicated that the data were compared with 24 hour saline treatment.

FIGS. 6A and 6A are photographs of representative untreated and HUCBC-treated rat MIs. Each row shows a series of sections of rat ventricles (from apex on the left to atrio-ventricular sulcus on the right). The upper rows of 6A and 6B portray untreated infarcts (32% of the total left ventricular area at 2 hr in FIG. 6A; 25% of the total area at 24 hr in FIG. 6B). The lower rows showing hearts treated with HUCBC at 2 hours (FIG. 6A with 1.8% of area) and 24 hours (FIG. 6B with 9% of area) show much less evidence of infarction.

FIG. 7 is a collection of four bar graphs, one each for TNF-α, MCP-1, MIP and INFγ. The cytokines/chemokines are compared for saline-treated and HUCBC-treated rat hearts. The myocardial concentrations of TNF-α, MCP-1, MIP and INFγ in HUCBC-treated rats did not significantly change during the initial 24 hours after coronary artery ligation, in contrast to the marked changes in saline-treated rats.

The following written description provides exemplary methodology and guidance for carrying out many of the varying aspects of the present invention.

### DETAILED DESCRIPTION

Determining the dosage and scheduling of a cellular therapy treatment regime is critical to developing an effective therapy that can be used to treat large numbers of patients. This is especially critical, when the disease affects a large number of patients within a given year, such as cardiovascular disease in general and acute myocardial infarction specifically. With approximately 1.2 million people in the USA experiencing a heart attack annually and approximately 500,000 people dying of the heart attack, the need for an effective treatment is evident. While stem cell therapy has shown promising results, determining the optimal time to dose a patient is one of the many aspects of this therapy that must be addressed. Previously, Saneron's collaborators discovered that administering cells to a stroked animal 48 hours after the stroke provide optimal results. However, when the experiment was conducted in an acute myocardial infarction animal model, the optimal timing was significantly different and appeared to have two optimal times, at 2 hours and 24 hours post attack.

This invention is intended to overcome, or at least alleviate, one or more of the difficulties or deficiencies associated with the prior art. In that regard, the present invention provides methods to enhance the therapeutic effects of cellular or drug treatment in various diseases and disorders. Preferably, the disorder is acute myocardial infarction.

In that regard, the present invention fulfills in part the need to identify new, unique methods for treating cardiovascular disease.

In one embodiment, the method comprises administering umbilical cord blood cells to an individual in need of treatment, wherein the umbilical cord blood cells are administered systemically to the individual at a time point specifically determined to provide therapeutic efficacy.

In one embodiment, the method comprises administering a therapeutic drug to an individual in need of treatment, wherein the drug is administered to the individual at a time point specifically determined to provide therapeutic efficacy.

In another embodiment, the optimal time to dose an individual in need of treatment is determined using a kit that measures the individual's blood for chemotactic factors and correlates the timing of treatment to the results of the kit.

Unless otherwise noted, the terms used herein are to be understood according to conventional usage by those of ordinary skill in the relevant art.

In addition to the definitions of terms provided below, definitions of common terms in molecular biology may also be found in Rieger et al., 1991 GLOSSARY OF GENETICS: CLASSICAL AND MOLECULAR, 5th Ed., Berlin: Springer-Verlag; and in CURRENT PROTOCOLS IN MOLECULAR BIOLOGY, F. M. Ausubel et al., Eds., Current Protocols, a joint venture between Greene Publishing Associates, Inc. and John Wiley & Sons, Inc. (1998 Supplement).

It is to be understood that as used in the specification and in the claims, “a” or “an” can mean one or more, depending upon the context in which it is used. Thus, for example, reference to “a cell” means at least one cell.

The present invention may be understood more readily by reference to the following detailed description of the preferred embodiments of the invention and the Examples included herein. However, before the present compounds, compositions, and methods are disclosed and described, it is to be understood that this invention is not limited to specific nucleic acids, specific polypeptides, specific cell types, specific host cells, specific conditions, or specific methods, etc., as such may, of course, vary, and the numerous modifications and variations therein will be apparent to those skilled in the art. It is also to be understood that the terminology used herein is for the purpose of describing specific embodiments only and is not intended to be limiting.

Standard techniques for cloning, DNA isolation, amplification and purification, for enzymatic reactions involving DNA ligase, DNA polymerase, restriction endonucleases and the like, and various separation techniques are those known and commonly employed by those skilled in the art. A number of standard techniques are described in Sambrook et al., 1989 MOLECULAR CLONING, Second Edition, Cold Spring Harbor Laboratory, Plainview, N.Y.; Maniatis et al., 1982 MOLECULAR CLONING, Cold Spring Harbor Laboratory, Plainview, N.Y.; Wu (Ed.) 1993 Meth. Enzymol. 218, Part I; Wu (Ed.) 1979 Meth Enzymol. 68; Wu et al., (Eds.) 1983 Meth. Enzymol. 100 and 101; Grossman and Moldave (Eds.) 1980 Meth. Enzymol. 65; Miller (ed.) 1972 EXPERIMENTS IN MOLECULAR GENETICS, Cold Spring Harbor Laboratory, Cold Spring Harbor, N.Y.; Old and Primrose, 1994 PRINCIPLES OF GENE MANIPULATION, 5th ed., University of California Press, Berkeley; Schleif and Wensink, 1982 Practical Methods in Molecular Biology; Glover (Ed.) 1985 DNA CLONING: VOLS. I AND II, IRL Press, Oxford, UK; Hames and Higgins (Eds.) 1985 NUCLEIC ACID HYBRIDIZATION, IRL Press, Oxford, UK; and Setlow and Hollaender 1979 GENETIC ENGINEERING: PRINCIPLES AND METHODS, Vols. 1-4, Plenum Press, New York City. Abbreviations and nomenclature, where employed, are deemed standard in the field and commonly used in professional journals such as those cited herein.

The present invention is also directed to a method of treating damage in the cardiovascular system which occurs as a consequence of genetic defect, physical injury, environmental insult or damage from a heart attack or cardiovascular disease in patients, the method comprising administering (including transplanting), an effective number, volume or amount of HUCBCs to patients at a time point specifically determined to provide optimal therapeutic efficacy.

In one embodiment, the administration of umbilical cord blood cells at a time point specifically determined to provide therapeutic efficacy leads to a determination that 2-3 days after an ischemic event monocyte chemoattractant protein-1 (MCP-1) expression is at its peak having been stimulated by IL-1, TNFα, IFNγ, LPS and platelet derived growth factor. MCP-1 is highly specific to monocytes and is expressed by endothelial cells and macrophages (Chen et al., 2001, ibid.; Yamagami et al., 1999, J Leukoc Biol 65:744-9). Prior to 48 hrs MCP-1 signals may not be strong enough to attract HUCBC and after 48 hrs it may be too late for cells in the ischemic core to recover.

The pharmaceutical compositions may further comprise a neural cell differentiation agent. The pharmaceutical compositions may further comprise a pharmaceutically acceptable carrier.

The term “patient” is used herein to describe an animal, preferably a human, to whom treatment, including prophylactic treatment, with the cells according to the present invention, is provided. For treatment of those conditions or disease states which are specific for a specific mammal such as a human patient, the term patient refers to that specific mammal.

The term “donor” is used to describe an individual (particularly a mammalian animal, including a human) who donates umbilical cord blood or umbilical cord blood cells for use in a recipient or patient.

The term “umbilical cord blood” (UCB) is used herein to refer to blood obtained from the umbilical cord and/or placenta, most preferably from a neonate. Preferably, the umbilical cord blood is isolated from human newborn umbilical cord and/or placenta. The use of umbilical cord blood as a source of mononuclear cells is advantageous because it can be obtained relatively easily and without trauma to the donor. In contrast, the collection of bone marrow cells from a donor is a traumatic experience. Umbilical cord blood cells (UCBCs) can be used for autologous transplantation or allogeneic transplantation, when and if needed. Umbilical cord blood is preferably obtained by direct drainage from the cord and/or by needle aspiration from the delivered placenta at the root and at distended veins.

Human umbilical cord blood is an extremely rich source of hematopoietic and mesenchymal progenitor cells. The total content of hematopoietic progenitor cells in umbilical cord blood equals or exceeds that in bone marrow, and the highly proliferative hematopoietic progenitor cells are eightfold higher in HUCBC than in bone marrow. The immunotype and functional properties displayed by human cord blood mesenchymal cells closely resembles the characteristics of bone marrow derived mesenchyman progenitor cells and mesenchymal and hematopoietic progenitor cells in umbilical cord blood can be expanded in tissue culture by as much as 77-95% (5, 6, 12). The percentage of primitive CD34 cells in umbilical cord blood decreases from 6 to 3% between gestation times of 17 weeks and 32 weeks and is approximately 1% for gestations of 37-41 weeks (34). HUCBC have been cryopreserved for as long as 15 years with recovery of 60-100% viable progenitor cells.

Cord blood mononuclear cells have longer telomeres than adult bone marrow cells, which indicates that HUCBC have undergone less cell division and are more immature than adult bone marrow stem cells. A distinct advantage of HUCBC over bone marrow stem cells is the immature immunogenicity of HUCBC, which are very similar to fetal stem cells, and which significantly reduces the risks of rejection by the transplant recipient or graft versus host disease in patients who are immunocompromised. In this regard, HUCBC T-lymphocytes are phenotypically and functionally naïve and generally have experienced little or no antigen exposure. HUCBC T-lymphocytes express predominantly a naïve form of CD45RA in contrast to human adult blood T cells that express a mature, memory isoform of CD45RO that is important in antibody production and cell proliferation. The majority of the CD45RA T cells that are expressed in cord blood do not produce CD40 ligand which is important in the B cell maturation. Consequently, umbilical cord blood B cells have intrinsic inabilities to produce immunoglobulins. The presence of the naïve form of CD45RA and the lack of CD40 ligand production contribute to the immature immunogenicity of HUCBC. In addition, HUCBC produce less cytokine IL-2, IL-3, INFγ, and TNFα than adult blood. For all these reasons human umbilical cord blood has been used as a source of marrow repopulating stem cells in patients treated for leukemia, myelodysplastic syndromes, neuroblastoma, Fanconi's anemia and aplastic anemia and more than 3000 human cord blood transplants have been performed in patients with these disorders.

We have recently begun to investigate the use of HUCBC as a source of stem cells for the treatment of acute myocardial infarction. In our initial study, we injected one million HUCBC directly into infracted myocardium of rats after permanent coronary occlusion and observed after one to three-four months the presence of HUCBC and infarction sizes in the HUCBC-treated rats that were significantly smaller than untreated infracted rat hearts. The reduction in infarction sizes in the HUCBC treated rats in our initial study was associated with LV ejection fractions and wall thickening, determined by echocardiography, that were 35% and 112% greater, respectively, than the untreated, infracted rats when measured at 4-5 months after the coronary occlusions and were similar to normal, unoperated control rats or the same age. Moreover, neither the HUCBC treated rats in our initial study nor the rats in the present study received any immunosuppressive therapy. Ion the present experiments (see below), the infarction sizes in rat hearts treated within two hr and also at 24 hr after coronary occlusion were more than 50-60% smaller than the infarction sizes in untreated rats. The results in the rats treated at 24 hr indicate that HUCBC can be administered relatively late after acute coronary occlusion and MI, in contract to primary thrombolytic therapy of primary angioplasty, and still produce substantial therapeutic effects. Human umbilical cord blood cells have also been administered for the treatment of acute stroke and traumatic brain injury in rats. In rats with stroke due to middle cerebral artery occlusion, HUCBC treatment at 24 hr after acute stroke decreased brain infarct volume by 65% in comparison with untreated rats. Moreover, the decrease in brain infarct volume was associated with a 25% increase in the rotarod test of physical agility and a 44% decrease in the modified Neurological Severity Score. Similarly in rats with traumatic brain injury, HUCBC treatment produced a 20% improvement in the rotarod test and a 55% decrease in the neurological severity scores. Taken together, these studies indicate that HUCBC can significantly limit the size of major organ infarctions.

As used herein, the term “human umbilical cord blood cells” (HUCBCs) refers to cells that are present within human umbilical cord blood and placenta. In one embodiment, the HUCBCs include a fraction of the UCB, containing mainly mononuclear cells that have been isolated from the umbilical cord blood using methods known to those skilled in the art. In a further embodiment, the HUCBCs may be differentiated prior to administration to a patient.

The term “effective amount” is used herein to describe concentrations or amounts of components such as differentiation agents, umbilical cord blood cells, precursor or progenitor cells, specialized cells, such as cardiomyocytes, or other agents which are effective for producing an intended result including differentiating stem and/or progenitor cells into specialized cells, such as cardiomyocytes, or heart attack, or accident victim or for effecting a transplantation of those cells within the patient to be treated. An effective amount can be determined for hypoxic neonates requiring high-dose oxygen therapy. Compositions according to the present invention may be used to effect a transplantation of the umbilical cord blood cells within the composition to produce a favorable change in the brain or spinal cord, or in the disease or condition being treated, whether that change is stabilization, an improvement (such as stopping or reversing the degeneration of a disease or condition being treated, such as reducing a neurological deficit or improving a neurological response) or a complete cure of the disease or condition treated.

The terms “stem cell” or “progenitor cell” are used interchangeably herein to refer to umbilical cord blood-derived stem and progenitor cells. The terms stem cell and progenitor cell are known in the art (e.g., STEM CELLS: SCIENTIFIC PROGRESS AND FUTURE RESEARCH DIRECTIONS, report from the National Institutes of Health, June, 2001).

The term “administration” or “administering” is used throughout the specification to describe the process by which cells of the subject invention, such as umbilical cord blood cells obtained from umbilical cord blood, or differentiated cells obtained therefrom, are delivered to a patient for therapeutic purposes. Cells of the subject invention are administered a number of ways including, but not limited to, parenteral, cardiac, intracardial, pericardial, intrathecal, intraventricular, intraparenchymal (including into the spinal cord, brainstem or motor cortex), intracistemal, intracranial, intrastriatal, and intranigral, among others. Basically any method can be used so that it allows cells of the subject invention to reach the ultimate target site. Cells of the subject invention can be administered in the form of intact umbilical cord blood or a fraction thereof (such term including a mononuclear fraction thereof or a fraction of mononuclear cells, including a high concentration of stem cells). The compositions according to the present invention may be used without treatment with a mobilization agent or differentiating agent (“untreated” i.e., without further treatment in order to promote differentiation of cells within the umbilical cord blood sample) or after treatment (“treated”) with a differentiation agent or other agent which causes certain stem and/or progenitor cells within the umbilical cord blood sample to differentiate into cells exhibiting a differentiated phenotype, such as a cardiomyocyte. The cells may undergo ex vivo differentiation prior to administration into a patient.

The umbilical cord blood stem cells can be administered systemically or to a target anatomical site, permitting the cells to differentiate in response to the physiological signals encountered by the cell (e.g., site-specific differentiation).

Administration often depends upon the disease or condition treated and may preferably be via a parenteral route, for example, intravenously, by administration into the pericardium or by direct implantation into the affected tissue in the heart.

The terms “grafting” and “transplanting” and “graft” and “transplantation” are used throughout the specification synonymously to describe the process by which cells of the subject invention are delivered to the site where the cells are intended to exhibit a favorable effect, such as repairing damage to a patient's myocardium (which can reduce the infarct size), treating an acute or subacute cardiovascular disease, physical injury, trauma, or environmental insult to the cardiovascular system, caused by, for example, an accident or other activity. Cells of the subject invention can also be delivered in a remote area of the body by any mode of administration as described above, relying on cellular migration to the appropriate area to effect transplantation.

The term “non-tumorigenic” refers to the fact that the cells do not give rise to a neoplasm or tumor. Stem and/or progenitor cells for use in the present invention are most preferably free from neoplastic and cancerous cells.

Acute myocardial infarction (AMI), Prinzmetal's angina pectoris and myocardial ischemia are caused by chronic and/or abrupt occlusion of major coronary arteries, usually caused by rupture of an existing atherosclerotic plaque. All may benefit from standard medical and surgical treatments and administration of HUCBCs to minimize inflammation and repair hypoxic/necrotic myocardial muscle tissue. An AMI generally occurs with the acute rupture of an atherosclerotic plaque causing activation of the blood clotting cascade leading to arterial occlusion, localized hypoxemia or anoxia and subsequent cell damage and/or death. In many instances, the localized area of infarction is extended peripherally through continued hypoxia and inflammatory processes. HUCBCs help repopulate necrotic myocardial muscle cells (i.e., dead cells) and to retard or reverse peripheral extension of the AMI. Prinzmetal's angina pectoris and myocardial ischemia are “chronic” myocardial ischemic conditions caused by slow occlusion, rather than acute occlusion of a cardiac artery. The ischemia associated with both draws administered HUCBCs to the affected site and help the patient by modifying the inflammatory responses and repopulating dysfunction cardiac cells. In vivo research has shown that administering HUCBCs in the time interval between 2 hours and 24 hours was optimal to obtain the maximal beneficial effect.

The term “gene therapy” is used throughout the specification to describe the transfer and stable insertion of new genetic information into cells for the therapeutic treatment of diseases or disorders. The foreign gene is transferred into a cell that proliferates to spread the new gene throughout the cell population. Thus, umbilical cord blood cells, or progenitor cells are the targets of gene transfer either prior to differentiation or after differentiation to a neural cell phenotype. The umbilical cord blood stem or progenitor cells of the present invention can be genetically modified with a heterologous nucleotide sequence and an operably linked promoter that drives expression of the heterologous nucleotide sequence. The nucleotide sequence can encode various proteins or peptides. The gene products produced by the genetically modified cells can be harvested in vitro or the cells can be used as vehicles for in vivo delivery of the gene products (i.e., gene therapy).

### Molecular Biology Techniques

Standard molecular biology techniques known in the art and not specifically described are generally followed as in Sambrook et al., MOLECULAR CLONING: A LABORATORY MANUAL, Cold Springs Harbor Laboratory, NY (1989, 1992), and in Ausubel et al., CURRENT PROTOCOLS IN MOLECULAR BIOLOGY, John Wiley and Sons, Baltimore, Md. (1989). Polymerase chain reaction (PCR) methodology is generally employed as specified as in Jam et al., PCR PROTOCOLS: A GUIDE TO METHODS AND APPLICATIONS, Academic Press, San Diego, Calif. (1999). Reactions and manipulations involving other nucleic acid techniques, unless stated otherwise, are performed as generally described in Sambrook et al., MOLECULAR CLONING: A LABORATORY MANUAL, Cold Springs Harbor Laboratory Press, and methodology as set forth in U.S. Pat. Nos. 4,666,828; 4,683,202; 4,801,531; 5,192,659; and 5,272,057, and incorporated herein by reference. In situ PCR in combination with flow cytometry can be used for detection of cells containing specific DNA and mRNA sequences (e.g., Testoni et al., 1996, Blood, 87:3822).

Standard methods in immunology known in the art and not specifically described herein are generally followed as set forth in Stites et al. (Eds.), BASIC AND CLINICAL IMMUNOLOGY, 8^{th} Ed., Appleton & Lange, Norwalk, Conn. (1994); and Mishell and Shigi (Eds.), SELECTED METHODS IN CELLULAR IMMUNOLOGY, W.H. Freeman and Co., New York (1980).

### Immunoassays

In general, immunoassays are employed to assess a specimen for cell surface markers or the like. Immunocytochemical assays are well known to those skilled in the art. Both polyclonal and monoclonal antibodies can be used in the assays. Where appropriate other immunoassays, such as enzyme-linked immunosorbent assays (ELISAs) and radioimmunoassays (RIAs), are well known to those skilled in the art and can be used. Available immunoassays are extensively described in the patent and scientific literature. See, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771; and 5,281,521 as well as Sambrook et al., MOLECULAR CLONING: A LABORATORY MANUAL, Cold Springs Harbor, N.Y., 1989. Numerous other references also may be relied on for these teachings.

### Antibody Production

Antibodies have attained wide use in the laboratory (as indicated in the following examples) and in clinical medicine. Conveniently, antibodies may be prepared against the immunogen or immunogenic portion thereof (for example, a synthetic peptide based on the sequence) or prepared recombinantly by cloning techniques or the natural gene product and/or portions thereof may be isolated and used as the immunogen. Immunogens can be used to produce antibodies by standard antibody production technology well known to those skilled in the art as described generally in Harlow and Lane, ANTIBODIES: A LABORATORY MANUAL, Cold Spring Harbor Laboratory, Cold Springs Harbor, N.Y. (1988) and Borrebaeck, ANTIBODY ENGINEERING—A PRACTICAL GUIDE by W.H. Freeman and Co., New York City (1992). Antibody fragments may also be prepared from the antibodies and include Fab and F(ab′)2 by methods known to those skilled in the art. To produce polyclonal antibodies a host, such as a rabbit or goat, is immunized with the immunogen or immunogenic fragment, generally with an adjuvant and, if necessary, coupled to an immunogenic carrier. Subsequently, antibodies specific to the immunogen are collected from the serum. Furthermore, the polyclonal antibody can be adsorbed such that it is monospecific. That is, the serum can be exposed to related immunogens so that cross-reactive antibodies are removed from the serum rendering it monospecific (i.e., the serum can be exposed to related immunogens so that cross-reactive antibodies are removed from the serum rendering the harvested antibodies).

To produce monoclonal antibodies, an appropriate donor (usually mammalian) is hyperimmunized with the immunogen, and splenic antibody-producing cells are isolated. These cells are fused to immortal cells, such as myeloma cells, to provide a fused hybrid cell line that is immortal and secretes the desired antibody. The cells are then cultured, and the monoclonal antibodies are harvested from the culture medium.

For producing recombinant antibodies, messenger RNA from antibody-producing B-lymphocytes of animals or hybridomas is reverse-transcribed to obtain complementary DNAs (cDNAs). Antibody cDNA, which encodes full or partial length antibody, is amplified and cloned into a phage or a plasmid. The cDNA can encode for be a partial length of heavy and light chain cDNA, separated or connected by a linker. The antibody, or antibody fragment, is expressed using a suitable expression system. Antibody cDNA can also be obtained by screening pertinent expression libraries. The antibody can be bound to a solid support substrate or conjugated with a detectable moiety or be both bound and conjugated as is well known in the art. (For a general discussion of conjugation of fluorescent or enzymatic moieties, see Johnstone & Thorpe, IMMUNOCHEMISTRY IN PRACTICE, 3d ed., Blackwell Scientific Publications, Oxford, 1996). The binding of antibodies to a solid support substrate is also well known in the art. (see for a general discussion Harlow & Lane, ANTIBODIES: A LABORATORY MANUAL, Cold Spring Harbor Laboratory Publications, New York, 1988; and Borrebaeck, ANTIBODY ENGINEERING—A PRACTICAL GUIDE, W.H. Freeman and Co., 1992). The detectable moieties contemplated with the present invention can include, but are not limited to, fluorescent, metallic, enzymatic and radioactive markers. Examples include biotin, gold, ferritin, alkaline phosphates, galactosidase, peroxidase, urease, fluorescein, rhodamine, tritium, ¹⁴C, iodination and green fluorescent protein.

### Gene Therapy

Gene therapy as used herein refers to the transfer of genetic material (e.g., DNA or RNA) of interest into a host to treat or prevent a genetic or acquired disease or condition. The genetic material of interest encodes a product (e.g., a protein, polypeptide, peptide, functional RNA, and/or antisense molecule) whose in vivo production is desired. For example, the genetic material of interest encodes a hormone, receptor, enzyme polypeptide or peptide of therapeutic value. Alternatively, the genetic material of interest encodes a suicide gene. For a review see “Gene Therapy” in ADVANCES IN PHARMACOLOGY, Academic Press, San Diego, Calif., 1997.

### Administration of Cells for Transplantation

The umbilical cord blood cells of the present invention can be administered and dosed in accordance with good medical practice, taking into account the clinical condition of the individual patient, the site and method of administration, scheduling of administration, patient age, sex, body weight and other factors known to medical practitioners. The pharmaceutically “effective amount” or dosage schedule for purposes herein is to be determined by such considerations as are known to those skilled in the experimental research, pharmacological and clinical medical arts. The amount must be effective to achieve stabilization, improvement (including but not limited to improved survival rate or more rapid recovery) or improvement or elimination of symptoms and other indicators as are selected as appropriate measures by those skilled in the art.

In the method of the present invention, the HUCBCs of the present invention can be administered in various ways as would be appropriate to implant in the central nervous system, including, but not limited to, parenteral administration, including intravenous and intraarterial administration, and pericardial administration.

Optionally, the HUCBCs are administered in conjunction with an immunosuppressive agent, such as cyclosporine.

[Pharmaceutical compositions comprising effective amounts of umbilical cord blood cells are also contemplated by the present invention. These compositions comprise an effective number of cells, optionally, in combination with a pharmaceutically acceptable carrier, additive or excipient and suspended in one or more appropriate liquid media. In certain aspects of the present invention, cells are administered to the patient in need of a transplant in sterile saline. In other aspects of the present invention, the cells are administered in Hanks Balanced Salt Solution (HBSS), Isolyte S, pH 7.4 or other such fluids chosen from 5% dextrose solution, 0.9% sodium chloride, or a mixture of 5% dextrose and 0.9% sodium chloride. Other examples of diluents are chosen from lactated Ringer's injection, lactated Ringer's plus 5% dextrose injection, Normosol-M and 5% dextrose, and acylated Ringer's injection. Still other approaches may also be used, including the use of serum free cellular media. Systemic administration of the cells to the patient may be preferred in certain indications; whereas, direct administration at the site of or in proximity to the diseased and/or damaged tissue may be preferred in other indications, as determined by the pharmaceutical presentation and as determined by those skilled in the art.

Pharmaceutical compositions according to the present invention preferably comprise an effective number of HUCBCs within the range of about 1.0×10⁴ cells to about 1.0×10¹⁴ cells, more preferably about 1×10⁵ to about 1×10¹³ cells, even more preferably about 2×10⁵ to about 8×10¹² cells, generally in suspension, optionally in combination with a pharmaceutically acceptable carrier, additives, adjuncts or excipients, as appropriate.

In one embodiment, HUCBCs are administered with cyclosporine or another anti-rejection compound.

Throughout this application, various publications are referenced. The disclosures of all of these publications and those references cited within those publications in their entireties are hereby incorporated by reference into this application in order to more fully describe the state of the art to which this invention pertains. The following examples are not intended to limit the scope of the claims to the invention, but are rather intended to be exemplary of certain embodiments. Any variations in the exemplified methods which occur to the skilled artisan are intended to fall within the scope of the present invention.

### EXAMPLES

### Materials and Methods

Acquisition of human umbilical cord blood cells (HUCBC). Cryopreserved (−196° C.) mononuclear fractions of HUCBC were obtained from the human cord cell blood bank (Saneron CCEL Therapeutics, Oldsmar, Fla.). Maternal blood was examined for HIV, HTLV, hepatitis, syphilis, and cytomegalovirus; and the cord blood was rejected is any test was positive. Cryopreserved HUCBC were thawed at 37° C. and transferred into a centrifuge tube containing Isolyte S, pH 7.4 (Braun McGaw). The cells were extensively washed, centrifuged at 1000 rpm for 7 min, the supernatant was discarded, and the HUCBC viability was determined. The HUCBC viability was 85-90% using the Trypan blue dye exclusion method. HUCBC volumes were adjusted to 1 amillion viable mononuclear cells/0.5 ml saline. The HUCBC contained 1.0-2.0% CD34⁺ cells as determined by flow cytometry.

Fluorescent Labeling of HUCBCs. The HUCBC were thawed at 37° C. and added to 7 mL Hank's Buffered Salt Solution (HBSS; Invitrogen, Carlsbad, Calif.). The sample was then centrifuged at 1100 rpm at 8° C. for a total period of 7 min. The resulting supernatant was removed and the cell pellet was resuspended in 800 μL Isolyte-S pH 7.4 (B Braun Medical) containing 100 μL DNase (Worthington Biochemical, Lakewood, N.J.). Next 20 μL of the HUCBC solution was removed, and the cell number and viability were determined using a cell counter (Beckman Coulter, Fullerton, Calif.). From a 1 mg/mL solution of 4′,6-diamidino-2-phenylindole (DAPI; Sigma-Aldrich, St. Louis, Mo.), 75 μL was removed and added to the cell solution, which was incubated on ice for 1 hr. Following incubation, the solution was centrifuged at 1200 rpm at 8° C. for 10 min. The resultant supernatant was decanted, and the cells were resuspended in 10 mL of phosphate buffered solution (PBS; Invitrogen). This resuspension and centrifugation step was repeated three more times. After the final decanting of 10 mL PBS, the cell pellet was resuspended in 1 mL Isolyte-S and placed on ice.

Normal and Infarcted Heart Extractions. All rats received care in compliance with the principles of laboratory animal care and in accordance with the University's Institutional Animal Care and Use Committee (IUCAC) and the Association for Assessment and Accreditation of Laboratory Animal Care International (AAALAC). Forty-five male Sprague-Dawley rats (Harlan, Indianapolis, Ind.) weighing between 150 and 200 g were anesthetized with 3-5% isoflurane by inhalation, intubated, and placed on mechanical ventilation with continuous isoflurane and oxygen. A left thoracotomy was performed through the fifth intercostals space. The pericardium was opened and the left anterior descending (LAD) artery was ligated. Infarction of the anterior wall of the left ventricle was confirmed by the presence of a paler myocardial color. The left ventricle of each heart was visually divided into a grid of nine equal sections and the number of sections occupied by the infarct were counted and recorded. The thoracotomy was then closed in three separate layers, and the rat was allowed to recover. Buprenorphine (0.05-0.5 mg/kg) was given for post-operative analgesia every six to eight hr. After periods varying of 1, 2, 2.5, 3, 6, 12, 24, 48 and 96 hr after the ligation, the rats were anesthetized with 60 mg/kg phenobarbitol administered intraperitoneally. The heart of each rat was rapidly excised and rinsed in physiological saline. The hearts were then flash frozen and stored in liquid nitrogen at −196° C. Normal hearts were obtained in the same manner; however, no surgical ligation of coronary vessels was performed.

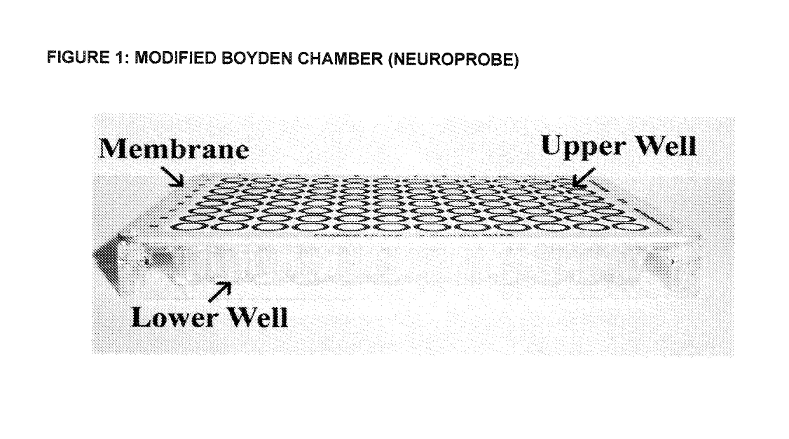

In Vitro Chemotactic Assay. Hearts were thawed at 37° C. The great vessels and atria were removed so that only ventricular tissue remained. The ventricular tissue was then weighed and placed in PBS at a concentration of 75 mg heart tissue per mL of PBS. The ventricular tissue in PBS was then homogenized until no tissue particles were visible in the solution. Then 300 μL of heart homogenate (normal and infracted from each heart) was placed into individual wells of a 96-well modified Boyden Chambers cell culture plate (Neuroprobe, Gaithersberg, Md.). A 25×80 mm framed polycarbonate membrane with 5 μm pore size (Neuroprobe) was placed on top of the lower chamber wells. This membrane has a pore density of 4,000/mm², with a pore area of 19.365 mm², and a pore area/unit area of 7.85%. The diameter of the HUCBC was 8±2 microns. (See FIG. 1.) Then in each corresponding upper well above heart homogenate, 10⁵ DAPI-fluorescent-labeled HUCBC in saline solution were placed. In addition, negative controls were set up in one row of wells in which the HUCBC were placed in upper wells but the corresponding bottom wells contained only suspension medium without heart homogenate, in order to determine and correct for random migration. Serial dilutions of DAPI labeled HUCBC from 0 to 80,000 were also placed in the first column of wells in each lower chamber for standard curve calibration purposes in order to compare cell fluorescence intensity to a known number of HUCBC. No HUCBC were placed in the first column of wells above these calibration cells.

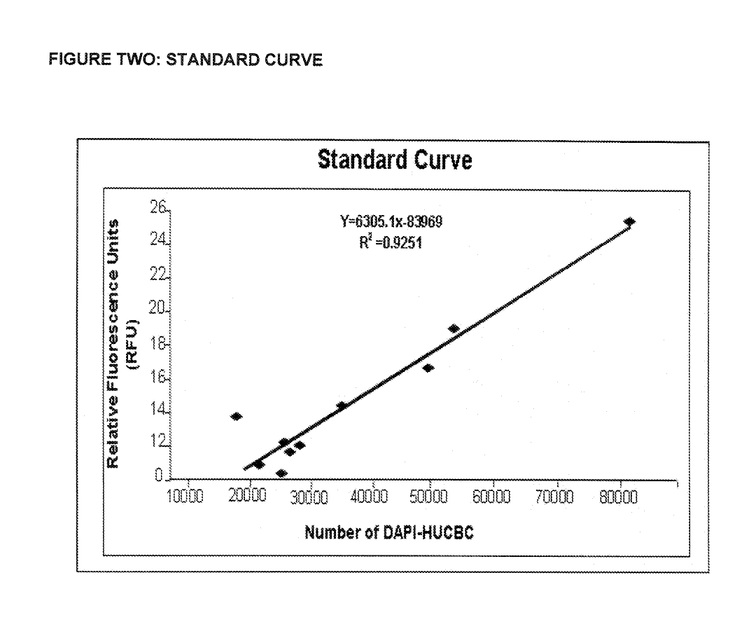

After incubation for 4 hr at 37° C. in a water-jacketed incubator with 5% CO₂ and air, the upper-wells were rinsed with PBS to remove any non-migrated cells. The plates were then centrifuged at 1200 rpm for 7 min to clear any partially migrated cells from the membrane into the lower wells. The upper-well plates and membrane were removed, and the lower-well plates were read in a fluorescent plate reader (Dynex MFX Microtiter, Chantilly, Va.) with a DAPI-specific filter set (absorption frequency of 358 nm and emission frequency of 461 nm) to determine HUCBC migration. The variability of the measurement technique was ≦±+5%. FIG. 2 shows a representative standard curve. Over the range between 0 and 80,000 cells, there is a linear relationship between the luminescent signals and the number of HUCBC (R²=0.93). The numbers of migrated cells for each of the five hearts at each time after LAD occlusion were determined and expressed as the mean ±SEM for each interval. Since the total number of migrated cells to each myocardial infarction may be determined, at least in part, by the size of the infarction, we divided each migration cell number by the total number of sections occupied by each infarct in order to adjust for infarction size.

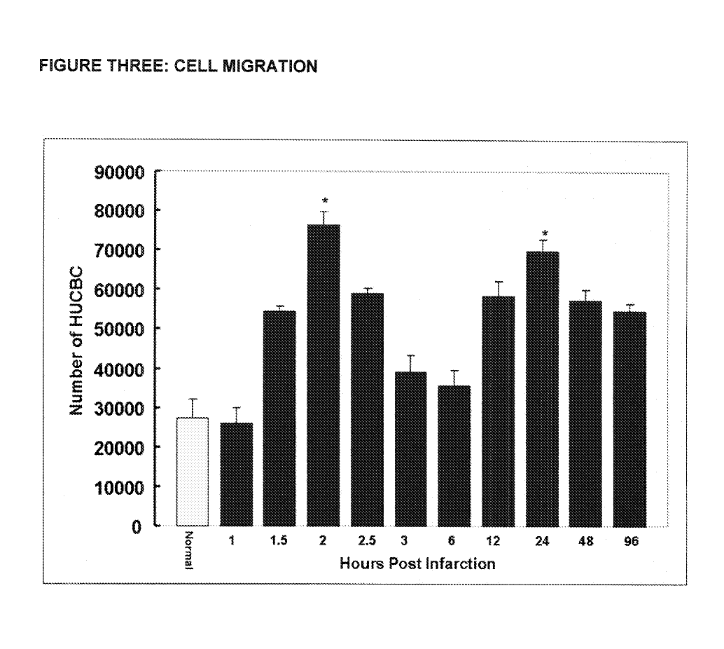

For results, we observed that during the period in the Boyden Chamber, 27.144±4968 DAPI-labeled HUCBC migrated to normal myocardium. The migration of HUCBC to infracted myocardium taken at 1 to 96 hr after coronary occlusion fluctuated and reached maximum numbers at 2 and at 24 hr. (See FIG. 3) A total of 76,331±3384 HUCBC migrated to infracted myocardium taken from rat hearts 2 hr after coronary occlusion and 69,911±2732 HUCBC migrated to infracted myocardium taken from rat hearts at 24 hr after LAD occlusion (both p<0.001 in comparison with normal hearts). HUCBC progenitor cells also migrated to infracted myocardium at the other times, but the cell number did not achieve statistically significant differences from the normal control myocardium at the other times (p≧0.05).

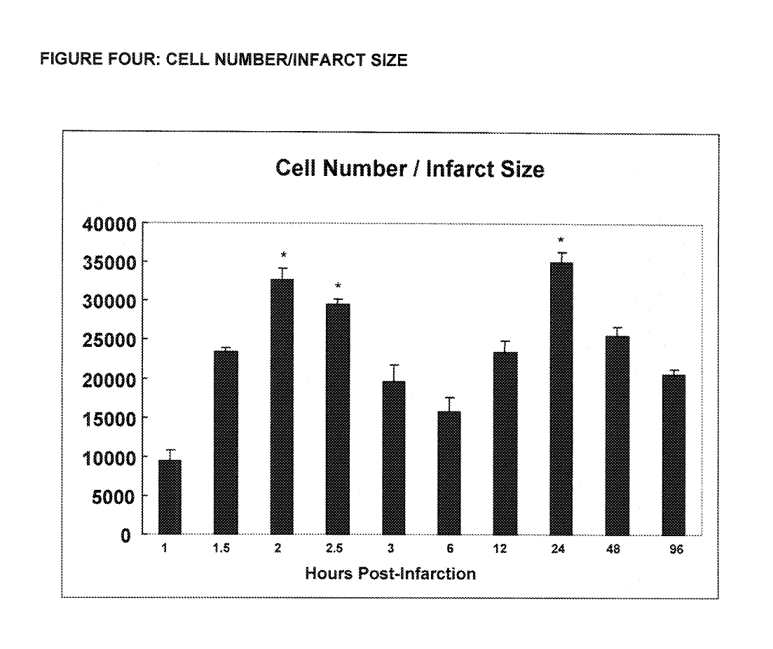

Because HUCBC migration to infracted tissue may increase with escalating sizes of myocardial infarction and increasing amounts of inflammation, the left ventricle of each heart had been divided into 9 sections to count the number of sections occupied by each MI. We then divided the cell migration number by the number of ventricular sections occupied by each MI. When the HUCBC migration number was corrected for the size of each MI, the fluctuation of HUCBC migration to infracted myocardium at each time was again evident. Significantly, the greatest HUCBC migration to infracted persisted at 2 and 24 hrs after LAD occlusion (p<0.01). (See FIG. 4). HUCBC migration to infracted myocardium was also significant (p<0.05) at 2.5 hr after acute coronary occlusion.

HUCBC Injection into Rats with Myocardial Infarctions. Thirty rats were anesthetized with 3-5% isoflurane and a left thoracotomy was performed. The pericardium was opened and the LAD permanently ligated. In ten rats, 106 HUCBC in 0.5 ml of saline were directly injected into the apex of the left ventricle within two hours after the coronary artery ligation. The injections were made into the apex of the left ventricle, which is the most muscular portion of the ventricle, in order to ensure direct myocardial distribution of HUCBC and also allow migration of HUCBC to the area of infarction in the anterior myocardial wall. In ten rats, only 0.5 ml of saline was injected into the apex of the left ventricle within two hours after the coronary artery ligation. In another ten rats, the LAD was ligated, and no injection was performed at that time. In order to quantitate the size of each infarction in each rat, the left ventricle of each heart was visually divided into a grid of nine equal sections and the number of sections occupied by each infarction counted and recorded. Each thoracotomy was then closed in three layers and each rat was allowed to recovered and given post-operative analgesia.

In the 10 rats with MIs that were not previously treated with injections, a second thoracotomy was performed 24 hr after the LAD occlusion. One million HUCBC in 0.5 ml of Isolyte pH 7.4 were then injected into the apex of the left ventricle in 5 rats and only 00.5 ml of saline in the other five rats. Each thoracotomy was then closed and the rats recovered with appropriate post-operative analgesia. No immunosuppressive drug therapy was given to any rat at any time. One month after the LAD occlusion, all rats were sacrificed and the hearts were extracted. The ventricles of each heart were cut into 2 mm slices parallel to the atrioventricular sulcus. Each ventricular slice was rinsed in saline and then immersed in 1% triphenyltetrazolium chloride (TTC) solution containing 0.2 M Tris. The myocardial slices were then rinsed in saline and photographed with a digital camera (Nikon). The heart slices were then stored in 10% formalin. Triphenyltetrazolium forms a red precipitate in the presence of intact dehydrogenase enzymes in normal myocardium; whereas, infracted and damaged myocardium lacks these enzymes and appears white to light pink in color within 30 min after acute coronary occlusion (1). Tetrazolium does not stain HUCBC but has a diagnostic efficiency of 88% for myocardial infarction (1). Computer imaging software (ImagePro Plus) was utilized for determination of the area of the infracted myocardium and the area of normal myocardium in the left ventricle.

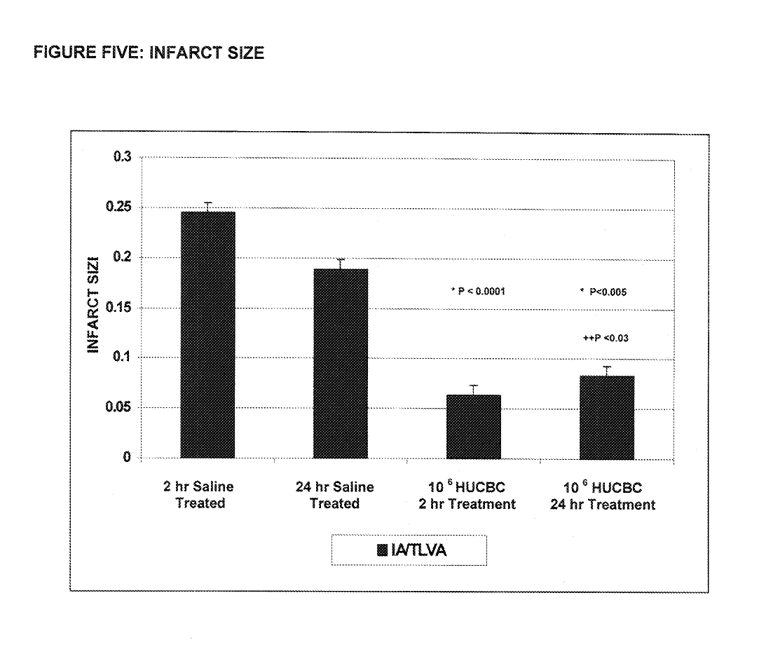

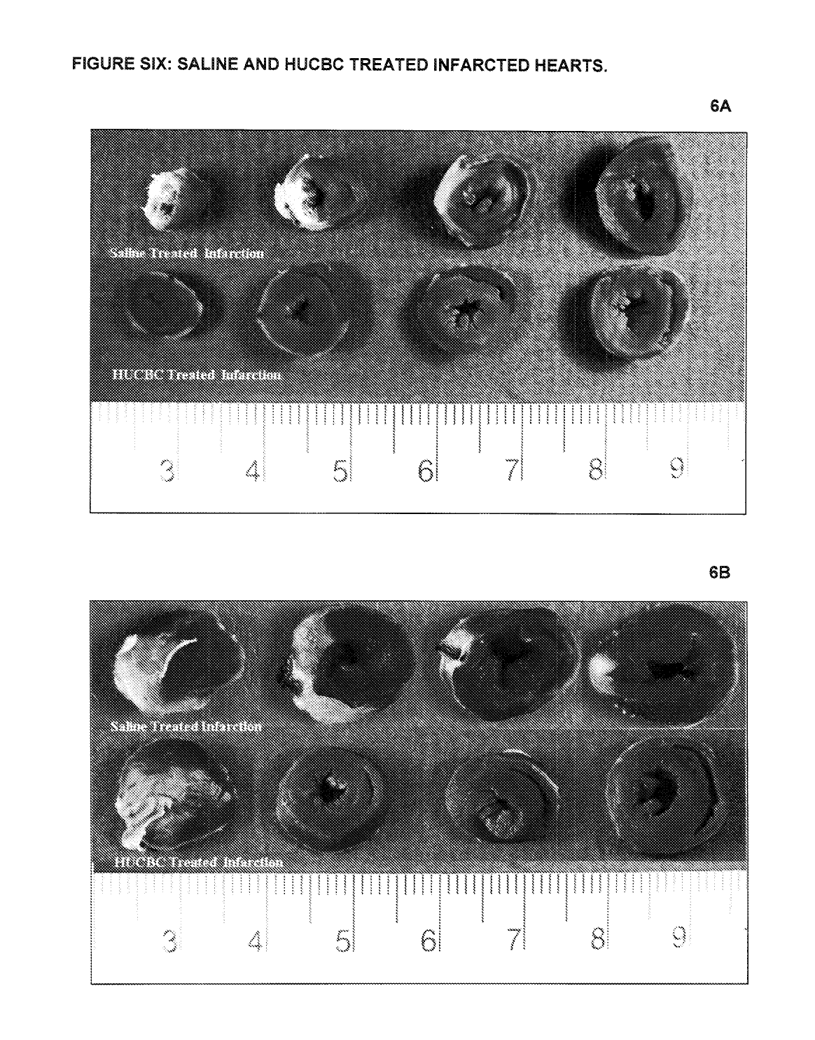

The myocardial infarctions following coronoary artery ligation occupied 2.5±0.5 out of 9 ventricular sections and there were no significant differences in infarct sizes between rats randomized to HUCBC treatment or saline treatment. Injection of 10⁶ HUCBC into infracted rat myocardium within 2 hr after acute coronary occlusion resulted in an infarct size at one month of 6.4±0.01% of the left ventricular muscle area (N=10) in comparison with infarct sizes of 24.5±0.02% of the left ventricular area (p<0.0001) in saline-treated infracted rat hearts. (See FIG. 5.) Human umbilical blood progenitor cells were also injected into infracted rat myocardium at 24 hr after acute coronary occlusion. Injection of 10⁶ HUCBC into infracted rat myocardium at 24 hr after acute coronary occlusion resulted in an infarct size in HUCBC-treated rats at one month of 8.4±0.02% of the total left ventricular area which was significantly less than the infracted hearts treated only with saline within 2 hr and at 24 hr. (See FIG. 5.) There was no myocardial histological evidence of rejection, such as lack of focal or diffuse inflammatory cell infiltrates when the hearts were examined at one month in the rats that received the HUCBC>Representative myocardial slices stained with tetrazolium from HUCBC-treated infracted hearts and saline-treated infracted hearts are shown in FIG. 6. In FIG. 6A in the representative 2 hr saline-treated heart, the infarct area occupied 32% of the total left ventricular area. In the 2-hr, HUCBC-treated heart, the infarct occupied 32% of the total left ventricular area. In the 2-hr, HUCBC-treated heart infarction, the infarct occupied 1.8% of the total left ventricular area. In FIG. 6B, the infarct in the 24-hr saline-treated heart occupied 25% of the total left ventricular area while the infarct in the 24-hr, HUCBC-treated heart occupied 9% of the total left ventricular area.

Infarct Sizing Experimental Procedure. Hearts were cut into 2.5 mm slices (6 slices per heart) parallel to the atrio-ventricular sulcus from the apex to the base. The 4 distal slices which correspond to the ventricular mass were immersed in a 15 triphenyltetrazolium chloride (TTC) solution containing 0.2 M Tris and incubated at 37° C. for 45 min. The heart slices were then rinsed in saline and stored in a 10% formalin solution for a period of 12 hr. With this tissue-staining protocol, infracted myocardial tissue appears white to light pink in color; whereas, normal myocardial tissue appears red in color. The heart slices were photographed using a digital camera, and ImagePro Plus 4.5 computer imaging software (Media Cybernetics, Silver Spring, Md.) was utilized for determination of damaged versus normal tissue. The portion of infarct area was calculated by dividing the infarct area by the total right and left ventricular area (TMA) and also by the left ventricular area alone (TLVA).

FIG. 6 shows relative infarct sizes for untreated (left) infarction, saline-injected treatment of infarct and infarct treated with 10⁶ HUCBC cells at one hr post ligation. Treatment with HUCBC at one hour resulted in a significantly smaller infarct (p<0.0001).

Cytokine and Chemokine Analysis. To determine the cytokines and chemokines present in farcted rat myocardium, a cytokine/chemokine antibody array kit (RayBiotech) that simultaneously profiles 18 cytokines and chemokines and permits comparison with known positive controls was used. Infarcted hearts injected with either HUCBC in saline or saline alone within 2 hr after coronary occlusion were extracted from rats at 2 hr (N=4), 6 hr (N=4), 12 hr (N=4) and 24 hr (N=6) after acute coronary occlusion. The hearts were flash frozen in liquid nitrogen and then stored at −86° C. The hearts were thawed at 25° C., and the left ventricle was dissected. The ventricular tissue was then placed in 750 μL of lysis buffer (20 mM Tris, pH 7.5, 0.3 M NaCl, 2% sodium deoxycholate, 2% TX-100 surfactant, plus a Protease Inhibitor Cocktail [Roche]) and homogenized until no visible tissue particles remained. The homogenate was then placed on a rocker plate at 4° C. for 2 hr and then centrifuged at 12000 RPM for 30 min at 4° C. The protein concentration of the supernatant was then determined by the Bradford Assay with bovine serum albumin as a standard. Fifty μg of protein from each heart supernatant was then added to 2 mL of blocking buffer (RayBiotech). Each solution was placed in a plastic try with a separate cytokine array membrane (Ray BioTech) and incubated for 3 hr at room temperature on a rocker plate. After incubation, the solution was removed and each membrane was incubated for 12 hr at 4° C. with a mixture of biotinylated cytokine primary antibodies to CINC2, CINC3, CNTF, Fractaline, GM-CSF, IL-1α, IL-1β, IL-4, IL-6, IL-10, LIX, IFNγ, leptin, MCP-1, MIP, βNGF, TNFα and VEGF (RayBiotech) on a rocker plate. The membranes were rinsed and HRP-conjugated secondary antibodies were added to each membrane. The membranes were incubated at room temperature for 2 hr, subjected to an enhanced chemiluminescence detection kit (Amersham) for 60 sec, and exposed on radiographic film (Amersham) for 90 sec. The blots on the radiographic film were then scanned and the protein densities determined with image analysis software (ImagePro). The results for each cytokine were then normalized to positive controls present on each membrane. Mean ±SEM values were then determined for each cytokine and chemokine at 2, 6, 12 and 24 hr after coronary occlusion.

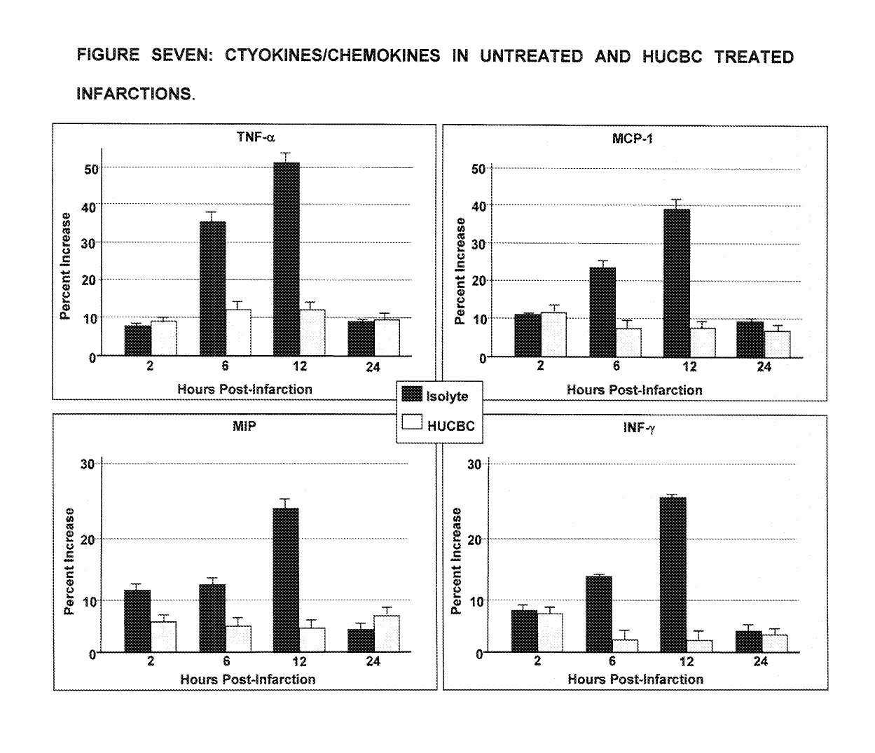

Acute MI in rat hearts treated with only saline produced two- to sevenfold increases in the ventricular myocardial tissue concentrations of tumor necrosis factor alpha (TNFα), monocyte/macrophage chemoattractant protein-1 (MCP-1), monocyte inflammatory protein (MIP), and interferon-gamma (INF-γ) between 2 and 12 hr after acute coronary occlusion. (See FIG. 7.) The increases in TNFα, MCP-1, MIP, and INF-γ in these rat hearts were most pronounced at 12 hr after acute coronary occlusion. TNFα increased from 6.9±0.8% to 51.2±4.6%, and INFγ increased from 8.9±0.3% to 25.0±1.7% (ANOVA all p<0.001). In contrast, the myocardial tissue concentrations of TNAα, MCP-1, MIP, and INF-γ in rat hearts treated with HUCBC did not significantly change at 2, 6, 12, or 24 hr after coronary occlusion. (See FIG. 7.)

Statistical Analyses. All result were expressed as the mean ±SEM. The differences between the controls and treated groups were tested by Dunnett's test. When multiple comparisons among groups were performed, analyses of variance were performed; then the Bonferroni modification of the t test was used for planned comparisons and Scheffe's procedure was used for post-hoc comparisons. A value of p<0.05 was considered significant.

### CONCLUSIONS

In the present investigation, infarcted myocardium attracted the largest number of HUCBC at 2 and 24 hr after coronary artery occlusion. Injection of one million HUCBC in saline into infracted rat hearts within 2 hr or at 24 hr after acute LAD occlusion resulted in MI sizes in these rats at 1 month that were more than 50% smaller than the infarct sizes in rats treated only with saline. Moreover, the myocardial concentrations of TNFα, MCP, MIP and INF-γ in the HUCBC-treated rat hearts did not significantly change from controls within the first 24 hr after acute coronary occlusion in contrast to two to sevenfold increases in these cytokines/chemokine in infracted rat hearts treated with only saline. The present investigations suggest that infracted myocardium significantly attracts HUCBC, that HUCBC can substantially reduce myocardial infarct size, and that HUCBC can limit inflammatory cytokine and chemokine expression in acutely infracted myocardium.

We used an in vitro chemotactic assay to determine the optimal time after myocardial infarction to transplant HUCBCs for myocardial repair. The greatest HUCBC migration occurred toward cell homogenate from MI hearts 2 and 24 hours after MI. A total of 76331±3384 HUCBCs migrated to infarcted myocardium at 2 hr and 69911±2732 at 24 hr after LAD occlusion (both p<0.001), and significantly exceeded HUCBC migration to normal hearts. Even when the cell migration number was corrected for infarct size/inflammatory response (graded 0 to 9), FIG. 4 shows that cell migration still remained greatest at 2/2.5 and 24 hours. We then injected 10⁶ HUCBCs into rat myocardial infarctions at these times, without using immunosuppression, and compared the sizes of the tetrazolium stained infarctions with untreated infarcted hearts at one month. When HUCBC were injected within 2 hr of acute LAD, the infarction size at 1 month was 7.6±4.6% (n=5) of ventricular muscle area, compared to untreated infarction sizes of 30.6±9.0% (n=5). We conclude that infarcted myocardium significantly attracts HUCBC, and HUCBC substantially reduce myocardial infarction size when promptly injected into infarcted hearts.

Chemotaxis, or cell locomotion directed towards an attractant, occurs during many biological processes including fertilization, embryonic development, hematopoiesis, tissue inflammation, and wound healing. A variety of methods have been devised to assay chamotaxis including migration of cells under an agarose layer, phagokinetic tract assays, cell orientation assays, time-lapse cinematography, and Boyden apparatus assays (14). The Boyden apparatus assay is a widely used method to measure chemotaxis (14). The chemotactic response with this assay can be analyzed by either manually measuring the distance traveled by cells or by visually quantifying the number of cells in the wells of the lower chamber of the Boyden apparatus. However, these methods are laborious and prone to subjective bias. Consequently, spectrophotometric, fluorescent or radiolabel techniques have been developed that provide precise quantification of cell migration in the Boyden apparatus. We therefore used a DAPI-fluorescent label for HUCBC and measured the fluorescence of the migrated cells. The fluorescent assay is not affected by filter pore size, cell density, filter composition, or filter thickness (14). With this technique, we were able to significantly limit variability in cell counts but detect substantial HUCBC migration to infracted myocardium. Using similar techniques, HUCBC also have been demonstrated to significantly migrate to ischemic and infracted cerebral tissue extracts taken at 24 hr after carotid occlusion and bone marrow stem cells have been demonstrated to migrate to ischemic cerebral extracts at 6, 24, and 48 hrs after carotid occlusion (9, 10, 28). In the preceding, we observed that the attraction of acutely ischemic/infracted myocardium for HUCBC fluctuates over time. This fluctuation in myocardial attraction for progenitor cells is most likely due to variations in the number or type of inflammatory cells and/or the concentrations of chemoattractants within the ischemic and infracted myocardium.

Chemokine and cytokine expression supported these conclusions. The present studies suggest that HUCBC limit TNFα, MCP-1 and MIP expression during the initial 24 hr after MI and may limit infarct size in this manner. Whereas, TNFα, MCP-1, MIP and INFγ were increased two- to sevenfold in infracted rat hearts treated with saline. This is similar to the finding that HUCBC treatment of acute strokes in rats significantly decreased the concentrations of TNFα and other cytokines; and this treatment was associated with significant decreases in infarct volume, the number of macrophages and B cells in the brain, and an increase in the functional recovery after stroke.

What is claimed is:

1. A method of treating acute myocardial infarction comprises providing human umbilical cord blood cells (HUCBC); and administering the HUCBC to the individual with the acute myocardial infarction at particular time intervals after said myocardial infarction.

2. The method of claim 1, wherein step b comprises administering the HUCBC at more than about one hour but less than about three hours after the acute myocardial infarction.

3. The method of claim 2, wherein the HUCBC are administered at about 2 hours after the acute myocardial infarction.

4. The method of claim 2, wherein the HUCBC are administered at about 2.5 hours after the acute myocardial infarction.

5. The method of claim 2, wherein step b comprises administering the HUCBC at about 1.5 hours after the acute myocardial infarction.

6. The method of claim 1, wherein step b comprises administering the HUCBC at more than about twelve hours but less than about forty-eight hours after the acute myocardial infarction.

7. The method of claim 6, wherein the HUCBC are administered at about 24 hours after the acute myocardial infarction.

8. The method of claim 1, wherein step b comprises administering HUCBC systemically.

9. The method of claim 8, wherein systemic administration is parenteral.

10. The method of claim 8, wherein the systemic administration is intravenous.

11. The method of claim 8, wherein the systemic administration is intracardiac.

12. The method of claim 8, wherein the systemic administration is intracoronary.

13. The method of claim 8, wherein the administration is within the coronary sac.

14. The method of claim 1, wherein the step of providing HUCBC further comprises providing a mononuclear fraction of HUCBC.
